(19) United States
(12) Patent Application Publication
(10) Pub. No.: US 2023/0245733 A1
(43) Pub. Date: Aug. 3, 2023

(54) METHOD AND SERVER FOR PROVIDING INTESTINAL MICROBIAL ANALYSIS RESULTS

(71) Applicant: HEM PHARMA INC., Pohang-si, Gyeongsangbuk-do (KR)

(72) Inventors: So Young PARK, Suwon-si, Gyeonggi-do (KR); Yo Sep JI, Suwon-si, Gyeonggi-do (KR)

(21) Appl. No.: 17/793,855

(22) PCT Filed: Dec. 15, 2020
(86) PCT No.: PCT/KR2020/018347

§ 371 Date: Dec. 8, 2022

(30) Foreign Application Priority Data
Jan. 23, 2020 (KR) 10-2020-0009521

(51) Int. Cl. G16H 10/40; G16H 10/20; G16H 10/60; G16H 50/20; G16H 70/60

(52) U.S. Cl. CPC G16H 10/40; G16H 10/20; G16H 10/60; G16H 50/20; G16H 70/60

(57) ABSTRACT

A method for providing an intestinal microbiome analysis result includes generating a plurality of groups based on previously analyzed intestinal microbiome information; receiving registration of a kit for collecting a user’s sample from a user device; classifying the user into at least one of the plurality of groups based on the user’s intestinal microbiome information obtained through an analysis of the user’s sample; and providing the user device with an intestinal microbiome analysis result including information about the at least one group into which the user is classified.

### TECHNICAL FIELD

The present disclosure relates to a method and server for providing an intestinal microbiome analysis result.

### BACKGROUND

Microbiota are known to play an important role in maintaining the homeostasis of the host (human) immunity, metabolites and the like. The microbiota and the host transmit and receive chemical signals to and from each other, and the expression of immune cells, production of neurotransmitter and production of short chain fatty acids (SCFA) by the microbiota have a significant effect on the host system.

Probiotics/ prebiotics balance the host’s unbalanced microbiota so that a healthy metabolite of the microbiota boosts the host’s health. Existing probiotics, like generic drugs, give everyone the same dose and similar species.

However, per-human microbiome similarity is less than 50%, and each person’s intestinal environment is different.

Meanwhile, even if a user examines the user’s intestinal environment through a stool test, it is difficult to check the state of the user’s intestinal environment because a test result is made up of terms difficult for the user to understand.

(Patent Document 1) Korean Patent Laid-open Publication No. 2019-0004586 (published on Jan. 14, 2019)

### DISCLOSURE OF THE INVENTION

### Problems to Be Solved by the Invention

The present disclosure is to solve the problems of the prior art described above, and to provide a user device with an intestinal microbiome analysis result including information about a group into which a user is classified based on the user’s intestinal microbiome information among a plurality of groups including a plurality of characteristics related to intestinal health.

The problems to be solved by the present disclosure are not limited to the above-described problems. There may be other problems to be solved by the present disclosure.

### Means for Solving the Problems

As a means for solving the problems, according to an aspect of the present disclosure, a method for providing an intestinal microbiome analysis result includes generating a plurality of groups based on previously analyzed intestinal microbiome information; receiving registration of a kit for collecting a user’s sample from a user device; classifying the user into at least one of the plurality of groups based on the user’s intestinal microbiome information obtained through an analysis of the user’s sample; and providing the user device with an intestinal microbiome analysis result including information about the at least one group into which the user is classified.

According to another aspect of the present disclosure, a server for providing an intestinal microbiome analysis result includes a group generation unit configured to generate a plurality of groups based on previously analyzed intestinal microbiome information; a kit registration unit configured to receive registration of a kit for collecting a user’s sample from a user device; a classification unit configured to classify the user into at least one of the plurality of groups based on the user’s intestinal microbiome information obtained through an analysis of the user’s sample; and an intestinal microbiome analysis result providing unit configured to provide the user device with an intestinal microbiome analysis result including information about the at least one group into which the user is classified.

The above-described aspects are provided by way of illustration only and should not be construed as liming the present disclosure. Besides the above-described embodiments, there may be additional embodiments described in the accompanying drawings and the detailed description.

### Effects of the Invention

According to any one of the above-described means for solving the problems of the present disclosure, the present disclosure can provide a user device with an intestinal microbiome analysis result, including information about a group into which a user is classified based on the user’s intestinal microbiome information among a plurality of groups including a plurality of characteristics related to intestinal health.

Accordingly, the present disclosure can help the user increase the understanding of the user’s intestinal environment by providing the user device with the intestinal microbiome analysis result of the user.

### BRIEF DESCRIPTION OF THE DRAWINGS

FIG. 1 is a configuration diagram of an intestinal microbiome analysis result providing system according to an embodiment of the present disclosure.

FIG. 2 is a block diagram of an intestinal microbiome analysis result providing server shown in FIG. 1 according to an embodiment of the present disclosure.

FIG. 3 is a diagram illustrating different character icons to be matched with a plurality of groups, respectively, according to an embodiment of the present disclosure.

FIG. 4A is a diagram for explaining a method for providing an intestinal microbiome analysis result according to an embodiment of the present disclosure.

FIG. 4B is a diagram for explaining the method for providing an intestinal microbiome analysis result according to an embodiment of the present disclosure.

FIG. 4C is a diagram for explaining the method for providing an intestinal microbiome analysis result according to an embodiment of the present disclosure.

FIG. 4D is a diagram for explaining the method for providing an intestinal microbiome analysis result according to an embodiment of the present disclosure.

FIG. 5 is a flowchart showing a method for providing an intestinal microbiome analysis result according to an embodiment of the present disclosure.

### BEST MODE FOR CARRYING OUT THE INVENTION

Hereafter, embodiments of the present disclosure will be described in detail with reference to the accompanying drawings so that the present disclosure may be readily implemented by a person with ordinary skill in the art. However, it is to be noted that the present disclosure is not limited to the embodiments but may be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “unit” includes a unit implemented by hardware, a unit implemented by software, and a unit implemented by both of them. One unit may be implemented by two or more pieces of hardware, and two or more units may be implemented by one piece of hardware.

Through the whole document, a part of an operation or function described as being carried out by a terminal or device may be carried out by a server connected to the terminal or device. Likewise, a part of an operation or function described as being carried out by a server may be carried out by a terminal or device connected to the server.

Hereinafter, the present disclosure will be explained in detail with reference to the accompanying configuration views or process flowcharts.

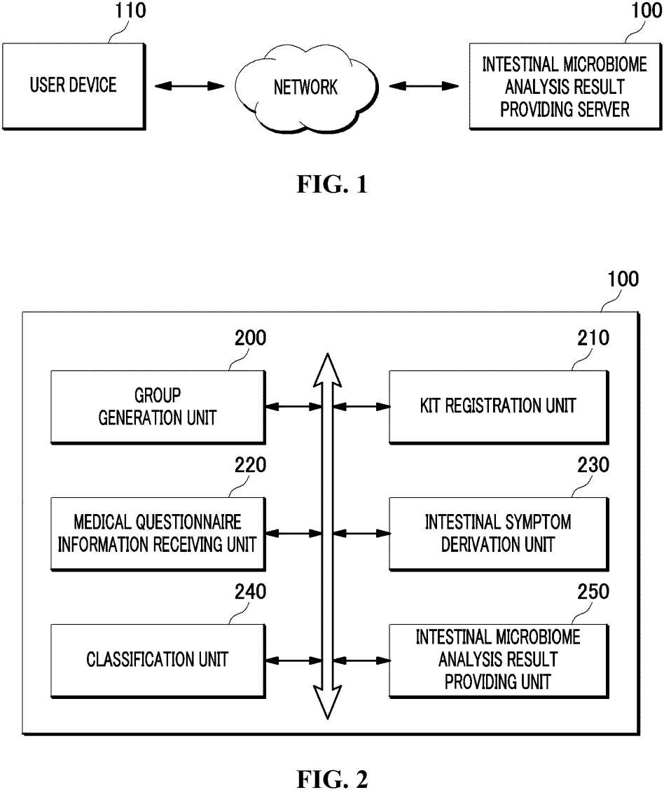

FIG. 1 is a configuration diagram of an intestinal microbiome analysis result providing system according to an embodiment of the present disclosure.

Referring to FIG. 1, an intestinal microbiome analysis result providing system may include an intestinal microbiome analysis result providing server 100 and a user device 110. However, the intestinal microbiome analysis result providing system illustrated in FIG. 1 is just an embodiment of the present disclosure and should not be construed as liming the present disclosure and can be configured differently from that shown in FIG. 1 according to various embodiments of the present disclosure.

The components of the intestinal microbiome analysis result providing system illustrated in FIG. 1 are typically connected to each other via a network (not shown). The network refers to a connection structure that enables information exchange between nodes such as devices and servers, and includes LAN (Local Area Network), WAN (Wide Area Network), Internet (WWW: World Wide Web), a wired or wireless data communication network, a telecommunication network, a wired or wireless television network, and the like. Examples of the wireless data communication network may include 3G, 4G, 5G, 3GPP (3rd Generation Partnership Project), LTE (Long Term Evolution), WIMAX (World Interoperability for Microwave Access), Wi-Fi, Bluetooth communication, infrared communication, ultrasonic communication, VLC (Visible Light Communication), LiFi, and the like, but may not be limited thereto.

The intestinal microbiome analysis result providing server 100 may generate a plurality of groups each including a plurality of characteristics related to intestinal health based on sample analysis and medical questionnaire information of a plurality of sample providers. That is, the intestinal microbiome analysis result providing server 100 may generate a group by grouping a plurality of sample providers having similar characteristics related to intestinal health based on a sample analysis and medical questionnaire information of the sample providers.

The user device 110 may access a personalized intestinal solution providing application or personalized solution providing web page in order to receive a personalized intestinal solution service, receive information about a kit to be used for examining a user’s intestinal environment from the user, and transmit the received information about the kit to the intestinal microbiome analysis result providing server 100. Herein, the kit is a tool used to collect the user’s sample. Herein, the sample is human feces.

When the user device 110 registers the kit for collecting the user’s sample in the intestinal microbiome analysis result providing server 100, the intestinal microbiome analysis result providing server 100 may classify the user into at least one of the plurality of groups based on the user’s intestinal microbiome information obtained through an analysis of the user’s sample.

The intestinal microbiome analysis result providing server 100 may provide the user device 110 with an intestinal microbiome analysis result including information about the at least one group into which the user is classified.

Hereinafter, the operation of each component of the intestinal microbiome analysis result providing system of FIG. 1 will be described in more detail.

FIG. 2 is a block diagram of the intestinal microbiome analysis result providing server 100 shown in FIG. 1 according to an embodiment of the present disclosure.

Referring to FIG. 2, the intestinal microbiome analysis result providing server 100 may include a group generation unit 200, a kit registration unit 210, a medical questionnaire information receiving unit 220, an intestinal symptom derivation unit 230, a classification unit 240 and an intestinal microbiome analysis result providing unit 250. However, the intestinal microbiome analysis result providing server 100 depicted in FIG. 2 is just one of embodiments of the present disclosure and can be modified in various ways based on the components depicted in FIG. 2.

The group generation unit 200 may generate a group by grouping a plurality of sample providers having similar characteristics related to intestinal health based on at least one of a sample analysis and medical questionnaire information of the sample providers. Herein, the sample analysis is an analysis to find out the type and distribution of the intestinal microbiome of a sample provider through a stool test of the sample provider. The medical questionnaire information may include a plurality of questions for checking the intestinal health conditions of the sample provider and the sample provider’s answers to the respective questions.

An analysis unit (not shown) may derive information about lifestyle, eating habit and defecation activity of each sample provider from the medical questionnaire information prepared by the plurality of sample providers.

The intestinal symptom derivation unit 230 may derive a plurality of symptoms related to intestinal health of each of the plurality of sample providers from the medical questionnaire information prepared by the plurality of sample providers. Herein, the plurality of symptoms related to intestinal health may include chronic abdominal distension, chronic abdominal pain, chronic diarrhea, chronic constipation, high body mass index (BMI) (obesity) symptoms and hyperglycemia.

The intestinal symptom derivation unit 230 may derive intestinal microbiome data associated with symptoms related to intestinal health of each sample provider from intestinal microbiome information obtained through an analysis of each sample provider’s sample.

For example, the intestinal symptom derivation unit 230 may derive the type and numerical information of the intestinal microbiome associated with an abdominal distension symptom based on intestinal microbiome information obtained through an analysis of samples from a plurality of sample providers who has abdominal distension symptoms. For example, if it is confirmed that Fusicatenibacter has a significant correlation with the abdominal distension symptoms, the intestinal symptom derivation unit 230 may divide a distribution of Fusicatenibacter into a section where a plurality of sample providers who answered they have abdominal distension symptoms belongs, a section where a plurality of sample providers who answered they have no symptom belongs and a section where sample providers belong regardless of symptoms.

The group generation unit 200 may semantically group distribution sections of the intestinal microbiomes associated with symptoms related to intestinal health and assign colors (e.g., gray, blue, yellow, red) and descriptions (e.g., the microbial level is appropriate, the microbial level is at an average level, the microbial level reaches a level requiring attention, the microbial level is out of detectable range) to respective groups.

The group generation unit 200 may generate a plurality of groups including a plurality of characteristics related to intestinal health based on information derived from at least one of the intestinal microbiome information obtained through an analysis of the plurality of sample providers’ samples or the medical questionnaire information of the plurality of sample providers.

The group generation unit 200 may select a character icon from among a plurality of character icons by age or lifestyle for each of the plurality of groups generated based on the intestinal microbiome information and the medical questionnaire information. Herein, the plurality of character icons characterizes the current status of the intestinal microbiome, and may be set based on at least one of the medical questionnaire information of the plurality of sample providers or the intestinal microbiome information. For example, the plurality of character icons may be created in consideration of age, lifestyle, eating habit and defecation activity of the plurality of sample providers.

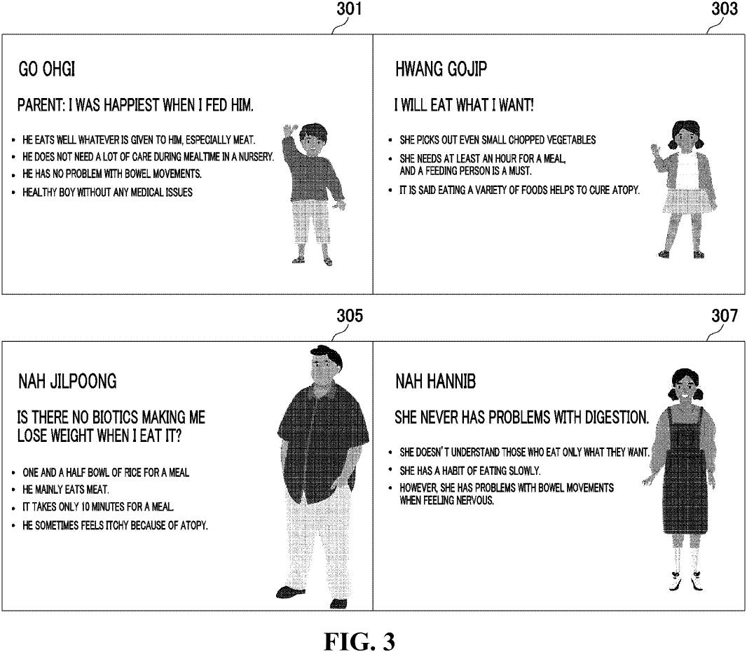

For example, referring to FIG. 3, the names of respective character icons 301, 303, 305 and 307 may be expressed with words that can infer the intestinal health conditions or lifestyle of sample providers belonging to the respective groups, and the character icons 301, 303, 305 and 307 may be expressed with images that represent body types depending on the age and eating habit of sample providers belonging to each group, and the characteristics of each character icon 301, 303, 305 or 307 may include information composed of at least one of lifestyle (e.g., with or without exercise, stress acceptance level, etc.), eating habit (e.g., habit of eating only what one wants, regular or irregular eating habit, favorite or disliked food information, etc.) and defecation activity (e.g., regular defecation, the frequency of constipation or diarrhea, etc.) of sample providers belonging to each group.

The group generation unit 200 may match a different character icon representing the characteristics of each group with each of the plurality of generated groups and generate information about each group based on information about the character icon matched with the group (e.g., name, image and characteristics of character).

The kit registration unit 210 may receive registration of a kit for collecting the user’s sample from the user device 110. Herein, the sample is human feces.

For example, the kit registration unit 210 may receive registration of information about a kit for examining the user’s intestinal environment from the user device 110 which has accessed the personalized intestinal solution providing application or personalized solution providing web page. For example, the kit registration unit 210 may receive, from the user device 110, a digital code (e.g., QR code, etc.) displayed on the kit scanned by a camera of the user device 110. Herein, the digital code may include information about the kit (e.g., identification information of the kit, etc.).

The medical questionnaire information receiving unit 220 may receive medical questionnaire information of the user from the user device 110. Herein, the medical questionnaire information may include a plurality of questions for checking the user’s intestinal health conditions, lifestyle and eating habit and the user’s answers to the respective questions.

The classification unit 240 may classify the user into at least one of a plurality of predetermined groups (groups matched with character icons representing the current status of the intestinal microbiomes) based on the intestinal microbiome information obtained through an analysis of the user’s sample. In this case, the classification unit 240 may classify the user into at least one of the plurality of groups based on a similarity between the user’s intestinal microbiome analysis information and intestinal microbiome analysis information corresponding to each of the plurality of groups.

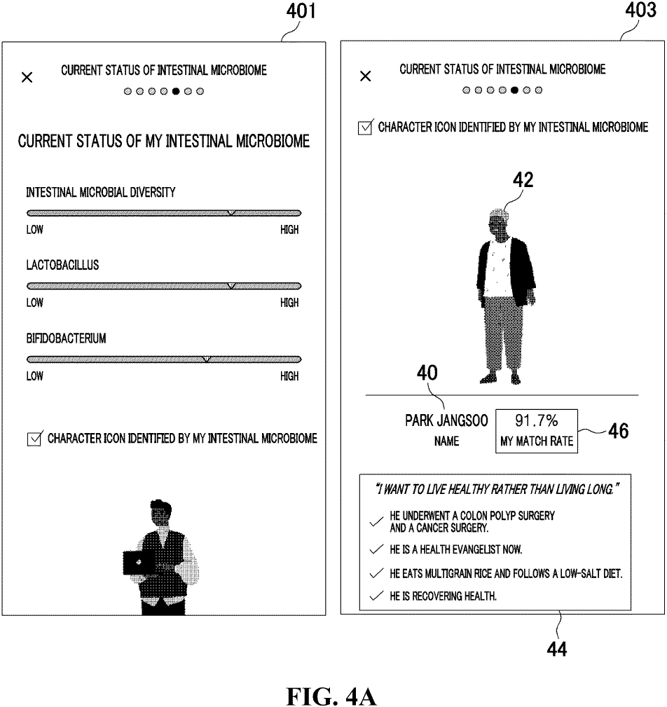

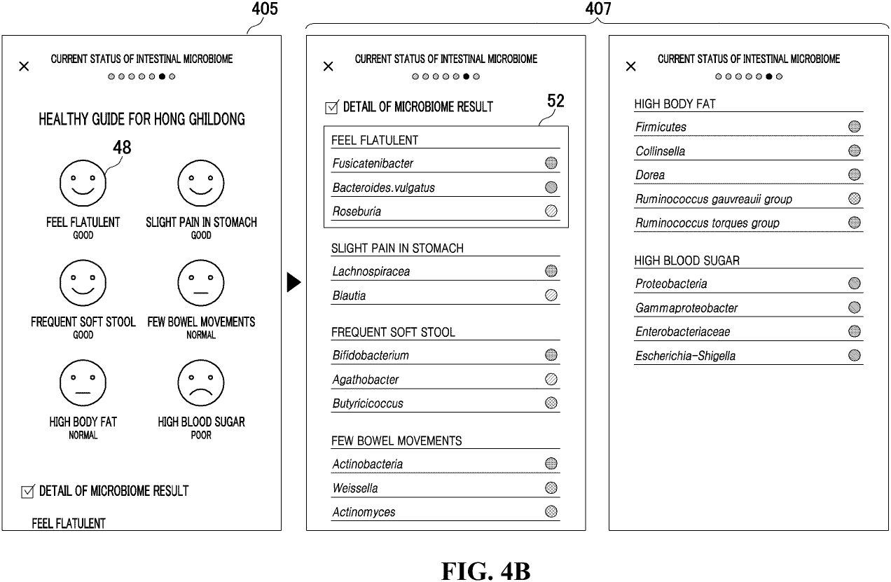

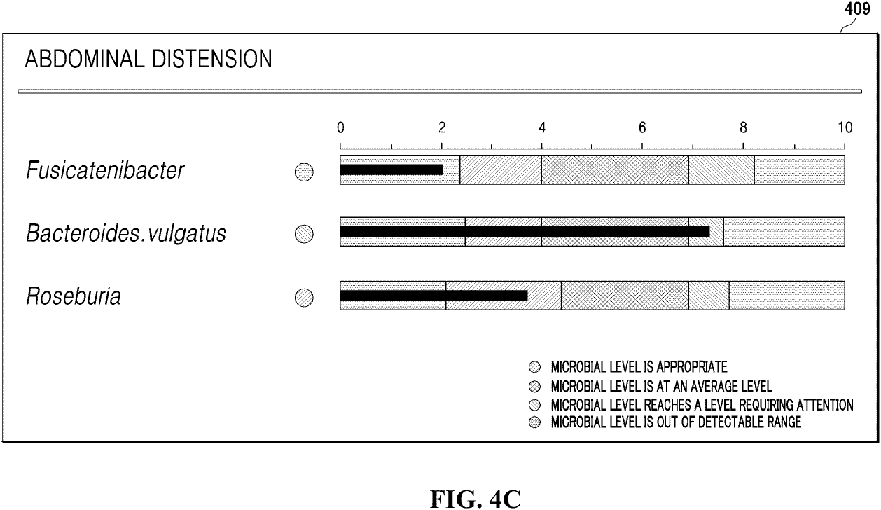

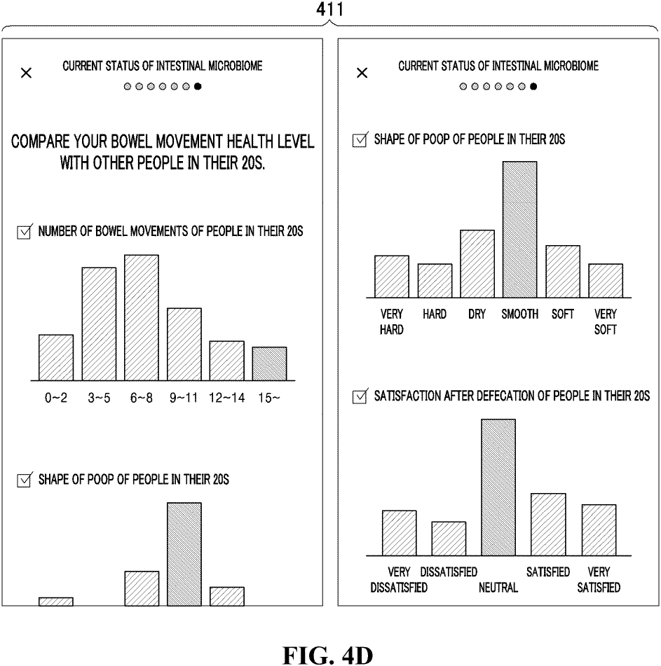

The intestinal microbiome analysis result providing unit 250 may provide the user device 110 with information about at least one group into which the user is classified. For example, referring to FIG. 4A, when the intestinal microbiome analysis result providing unit 250 receives a request for a character icon identified by the user’s intestinal microbiome from the user device 110 on the screen displaying intestinal microbiome status information 401, the intestinal microbiome analysis result providing unit 250 may provide the user device 110 with information 403 about a group including a name 40, an image 42 and characteristics 44 of a character icon representing the group to which the user belongs.

Also, the intestinal microbiome analysis result providing unit 250 may provide the user device 110 with the information 403 about the group including a match rate 46 between intestinal microbiome information corresponding to the group to which the user belongs and the user’s intestinal microbiome information. Accordingly, the present disclosure can help the user increase the understanding of the user’s intestinal environment by providing the user with the user’s intestinal microbiome information in the form of a character icon.

The intestinal microbiome analysis result providing unit 250 may provide the user device 110 with the user’s intestinal microbiome information derived through the analysis of the user’s sample. Herein, the user’s intestinal microbiome information may include, for example, intestinal microbial diversity information, Lactobacillus distribution information, Bifidobacterium distribution information, and the like. Herein, with respect to the intestinal microbial diversity information, how many different microbes are present in the human intestine is an important criterion for healthy intestine, and an decrease of the intestinal microbiome is related to irritable colitis, inflammatory bowel diseases, colorectal cancer, and the like. Lactobacillus is a beneficial bacterium present in many fermented foods and inhibits the growth of harmful bacteria in the intestine, and Bifidobacterium is a representative beneficial bacterium in the intestine and produces a large amount of beneficial substances to make intestinal cells healthy.

For example, referring to FIG. 4A, the intestinal microbiome analysis result providing unit 250 may provide the user device 110 with the intestinal microbiome status information 401 including the user’s intestinal microbial diversity information (i.e., information about which stage a score for the user’s intestinal microbial diversity belongs to among a plurality of stages (e.g., low-medium-high)), the amount of Lactobacillus and the amount of Bifidobacterium through the personalized intestinal solution providing application or personalized solution providing web page.

The intestinal microbiome analysis result providing unit 250 may provide the user device 110 with an intestinal microbiome analysis result including information about at least one group to which the user belongs.

Referring to FIG. 4B and FIG. 4C together, the intestinal microbiome analysis result providing unit 250 may provide a plurality of symptom results 405 based on the user’s intestinal microbiome analysis result for each of a plurality of symptoms related to intestinal health (chronic abdominal distension, chronic abdominal pain, chronic diarrhea, chronic constipation, high body mass index (BMI) (obesity) symptoms and hyperglycemia). In this case, the user’s intestinal microbiome analysis result may include emoticon facial expression information representing the intestinal conditions for each of the plurality of symptoms. In this case, the emoticon facial expression information may be expressed differently for each stage of the user’s intestinal conditions (e.g., good stage, normal stage, poor stage) depending on each symptom.

The intestinal microbiome analysis result providing unit 250 may provide the user device 110 with information about each of the plurality of symptoms related to the user’s intestinal health and an analysis result 407 of at least one intestinal microbiome associated with each symptom. For example, when one 48 of the plurality of symptom results 405 based on the user’s intestinal microbiome analysis result is selected by the user device 110, the intestinal microbiome analysis result providing unit 250 may provide the user device 110 with information about at least one intestinal microbiome associated with the selected symptom result 48.

The intestinal microbiome analysis result providing unit 250 may classify the intestinal microbiome analysis result including the amount of each intestinal microbiome of the user into at least one of a plurality of predetermined groups (groups matched with sections of the intestinal microbiomes significantly associated with respective syndromes and provide it to the user device 110. Referring to reference numeral 52 of FIG. 4B, it can be seen that among the microbiomes associated with chronic abdominal distension, Fusicatenibacter is in a gray group (regardless of symptoms), Bacteroides. vulgatus is in a red group (positively correlated with symptoms), and Roseburia is in a blue group (negatively correlated with symptoms).

Further, when the user device 110 selects the name of the intestinal microbiome from information 52 about at least one intestinal microbiome associated with the selected symptom result 48, the intestinal microbiome analysis result providing unit 250 may provide the user device 110 with the amount of the intestinal microbiome associated with the corresponding symptom.

For example, when the user selects the information 52 about at least one intestinal microbiome associated with chronic abdominal distension, the intestinal microbiome analysis result providing unit 250 may provide the user device 110 with a degree of distribution 409, which indicates how much at least one of Fusicatenibacter, Bacteroides.vulgatus and Roseburia associated with chronic abdominal distension is distributed in the intestine of the user, in the form of a graph (see FIG. 4C). Herein, the graph is expressed using a plurality of predetermined group colors. Accordingly, the present disclosure can help the user increase the understanding of the user’s intestinal environment by providing the user with the user’s intestinal microbiome information connected to a plurality of symptoms in various ways.

Referring to FIG. 4D, the intestinal microbiome analysis result providing unit 250 may provide the user device 110 with statistics on a plurality of bowel movement health levels (e.g., statistics on each of the average number of bowel movements for a predetermined period of time, the shape of poop and satisfaction after defecation) to compare the user’s bowel movement health level depending on the user’s defecation activity with that of his or her age group. Herein, the statistics on the plurality of bowel movement health levels may vary depending on the collection period or collection amount of the medical questionnaire information of the plurality of sample providers.

The group generation unit 200 may additionally reflect intestinal health characteristics of a group similar in intestinal microbiome information to the user among a plurality of groups to the user’s intestinal microbiome analysis result provided to the user device 110. Also, the group generation unit 200 may subdivide the plurality of generated groups based on intestinal microbiome analysis results of a plurality of users, and, thus, intestinal health characteristics of each group can be elaborated.

Meanwhile, it would be understood by a person with ordinary skill in the art that each of the group generation unit 200, the kit registration unit 210, the medical questionnaire information receiving unit 220, the intestinal symptom derivation unit 230, the classification unit 240 and the intestinal microbiome analysis result providing unit 250 can be implemented separately or in combination with one another.

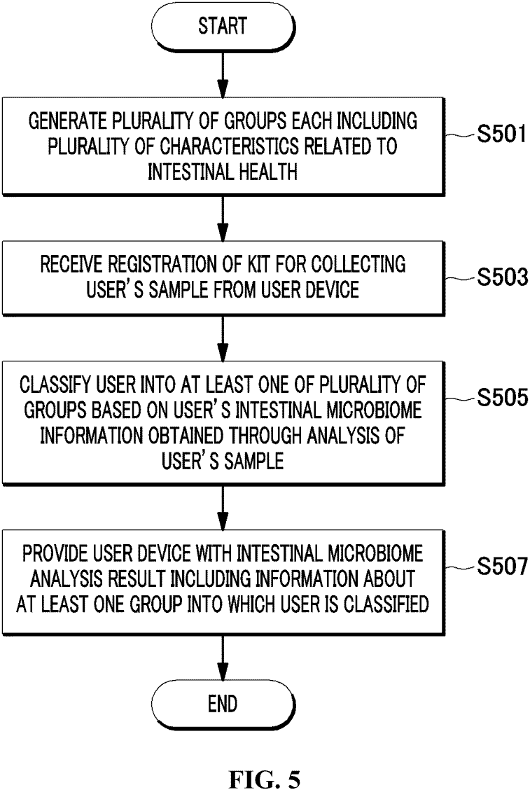

FIG. 5 is a flowchart showing a method for providing an intestinal microbiome analysis result according to an embodiment of the present disclosure.

Referring to FIG. 5, in a process S501, the intestinal microbiome analysis result providing server 100 may generate a plurality of groups each including a plurality of characteristics related to intestinal health based on intestinal microbiome information obtained through an analysis of samples from a plurality of sample providers and medical questionnaire information of the plurality of sample providers. Herein, each of the plurality of groups is represented by a different character icon, and information about the group including the image and characteristics (including lifestyle, eating habit and defecation activity information) of the character icon representing each group can be mapped to each group.

In a process S503, the intestinal microbiome analysis result providing server 100 may receive registration of a kit for collecting the user’s sample from the user device 110.

In a process S505, the intestinal microbiome analysis result providing server 100 may classify the user into at least one of the plurality of groups based on the user’s intestinal microbiome information obtained through an analysis of the user’s sample.

In a process S507, the intestinal microbiome analysis result providing server 100 may provide the user device 110 with an intestinal microbiome analysis result including information about the at least one group into which the user is classified. Herein, the information about the at least one group into which the user is classified may include an image and characteristics of a character icon representing the at least one group into which the user is classified.

In the descriptions above, the processes S501 through S507 may be divided into additional processes or combined into fewer processes depending on an exemplary embodiment. In addition, some of the processes may be omitted and the sequence of the processes may be changed if necessary.

An aspect of the present disclosure can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage medium includes all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. A method for providing an intestinal microbiome analysis result, comprising:
generating a plurality of groups based on previously analyzed intestinal microbiome information;
receiving registration of a kit for collecting a user’s sample from a user device;
classifying the user into at least one of the plurality of groups based on the user’s intestinal microbiome information obtained through an analysis of the user’s sample; and
providing the user device with an intestinal microbiome analysis result including information about the at least one group into which the user is classified.

2. The method for providing an intestinal microbiome analysis result of claim 1,
wherein the plurality of groups is generated based on intestinal microbiome information obtained through an analysis of samples from a plurality of sample providers and medical questionnaire information of the plurality of sample providers.

3. The method for providing an intestinal microbiome analysis result of claim 1,
wherein each of the plurality of groups is represented by a different character icon, and
the information about the at least one group into which the user is classified includes an image and characteristics of a character icon representing the at least one group into which the user is classified.

4. The method for providing an intestinal microbiome analysis result of claim 3,
wherein the information about the at least one group further includes a match rate between intestinal microbiome information corresponding to the group into which the user is classified and the user’s intestinal microbiome information.

5. The method for providing an intestinal microbiome analysis result of claim 3,
wherein the characteristics of the character icon includes at least one of lifestyle, eating habit and defecation activity.

6. The method for providing an intestinal microbiome analysis result of claim 1,
wherein the classifying the user into at least one of the plurality of groups includes:
classifying the user into at least one of the plurality of groups based on a similarity between the user’s intestinal microbiome information and intestinal microbiome information corresponding to each of the plurality of groups.

7. The method for providing an intestinal microbiome analysis result of claim 1, further comprising:
receiving medical questionnaire information of the user from the user device.

8. The method for providing an intestinal microbiome analysis result of claim 7, further comprising:
deriving a plurality of symptoms related to intestinal health of the user based on the medical questionnaire information.

9. The method for providing an intestinal microbiome analysis result of claim 8,
wherein the intestinal microbiome analysis result further includes information about the plurality of symptoms and information about at least one of intestinal microbiomes that cause the plurality of symptoms, respectively.

10. The method for providing an intestinal microbiome analysis result of claim 1,
wherein the intestinal microbiome analysis result further includes the amount of each intestinal microbiome of the user and statistics on a plurality of bowel movement health levels.

11. A server for providing an intestinal microbiome analysis result, comprising:
a group generation unit configured to generate a plurality of groups based on previously analyzed intestinal microbiome information;
a kit registration unit configured to receive registration of a kit for collecting a user’s sample from a user device;
a classification unit configured to classify the user into at least one of the plurality of groups based on the user’s intestinal microbiome information obtained through an analysis of the user’s sample; and
an intestinal microbiome analysis result providing unit configured to provide the user device with an intestinal microbiome analysis result including information about the at least one group into which the user is classified.

12. The server for providing an intestinal microbiome analysis result of claim 11,
wherein each of the plurality of groups is represented by a different character icon, and
the information about the at least one group into which the user is classified includes an image and characteristics of a character icon representing the at least one group into which the user is classified.

13. The server for providing an intestinal microbiome analysis result of claim 12,
wherein the information about the at least one group further includes a match rate between intestinal microbiome information corresponding to the group into which the user is classified and the user’s intestinal microbiome information.

14. The server for providing an intestinal microbiome analysis result of claim 11,
wherein the classification unit is configured to classify the user into at least one of the plurality of groups based on a similarity between the user’s intestinal microbiome information and intestinal microbiome information corresponding to each of the plurality of groups.

15. The server for providing an intestinal microbiome analysis result of claim 11, further comprising:
a medical questionnaire information receiving unit configured to receive medical questionnaire information of the user from the user device.

16. The server for providing an intestinal microbiome analysis result of claim 15, further comprising:
an intestinal symptom derivation unit configured to derive a plurality of symptoms related to intestinal health of the user based on the medical questionnaire information.

17. The server for providing an intestinal microbiome analysis result of claim 16,
wherein the intestinal microbiome analysis result further includes information about the plurality of symptoms and information about at least one of intestinal microbiomes that cause the plurality of symptoms, respectively.
